(19) United States
(12) Patent Application Publication
(10) Pub. No.: US 2007/0092869 A1
(43) Pub. Date: Apr. 26, 2007

(54) Spike-in controls and methods for using the same

(76) Inventors: Stephanie B. Fulmer-Smentek, Cupertino, CA (US); Anne Bergstrom Lucas, Mountain View, CA (US); Diane D. Ilsley, Dripping Springs, TX (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,675

(22) Filed: Oct. 13, 2006

Related U.S. Application Data
(60) Provisional application No. 60/730,163, filed on Oct. 24, 2005.

(51) Int. Cl. C12Q 1/70; C12Q 1/68; G06F 19/00

(52) U.S. Cl. 435/5; 702/20; 435/6

(57) ABSTRACT

Methods, compositions, and kits for performing a one-color analysis of microarray data are provided. Also disclosed are compositions including control nucleic acid sequences, and methods for measuring the dynamic range of a microarray analysis.

### CROSS-REFERENCE TO RELATED APPLICATIONS

This is a nonprovisional application based on U.S. Provisional Application No. 60/730,163, filed Oct. 24, 2005, and is related to copending nonprovisional U.S. patent application Ser. No. (serial number unknown), entitled “Estimation Of Dynamic Range Of Microarray DNA Spike-In Data By Use Of Parametric Curve Fitting” filed Oct. 4, 2006, the disclosure of which is incorporated herein by reference in its entirety.

### BACKGROUND

Gene expression analysis can be performed by one-color (intensity-based) or two-color (ratio-based) microarray platforms depending on the specific applications. A one-color procedure involves the hybridization of a single sample to each microarray after it has been labeled with a single fluorophore, whereas in a two-color experiment, two samples (experimental and control) are labeled with different fluorophores and hybridized to a single microarray.

The two-color approach was developed to reduce errors associated with microarray manufacture, but this is less of a problem with the availability of high quality commercial microarrays with decreased variability. The principal advantage of the two-color system is that it allows direct comparison between two samples on a microarray, minimizing errors due to experimental microarray processing. However, dye-specific biases can significantly impact results when using the two-color approach. While these biases can be mitigated via dye-swapping, or fluorophore reversals, the technical replication required adds to the experimental costs of a microarray analysis.

A one-color approach, when paired with high quality microarrays and a robust workflow, offers more flexibility in experimental design with the possibility of reduced error in complex comparisons. Hybridization of a single sample per microarray (rather than two samples) facilitates comparisons across microarrays and between groups of samples. Data variability across assays due to multiple sources of variability, including microarray fabrication and processing, can be reduced for one-color methods, by performing sufficient biological or technical replicate assays. Two of the major requirements of a one-color microarray platform are: the ability to conduct high confidence experiments with reliable system reproducibility, and the ability to detect significant gene expression changes through high sensitivity.

### SUMMARY

Compositions and methods for measuring dynamic range of one-color microarray assays for gene expression are described herein. In certain aspects, the method involves using compositions known as spike-in controls, (hereinafter “spike-ins”) i.e., compositions that can be added to a sample of target nucleic acids being analyzed to allow a user to assess any degradation in the overall performance of the microarray (including, but not limited to signal to noise, dynamic range, linearity of response, and background). In one aspect, spike-in control compositions are provided. These compositions comprise a set of defined sequence nucleic acids that bind or hybridize, under the employed hybridization conditions, to control probes with complementary sequences at pre-defined positions within one or more arrays. In one aspect, a plurality of control probe features is provided on an array, those probes being complementary to a plurality of spike-in controls. The plurality of spike-ins comprise defined sequences each present at a different known relative concentration; the complexes formed between control probe molecules and spike-ins are present in the same relative ratios across the different sequences, thereby providing an assessment of the performance of the arrays. In one aspect, a spike-in composition includes a subsequence of a complementary adenovirus type 5 sequence, such as a subsequence of the E1A sequence, in addition to the defined sequence to which the probe is complementary.

Compositions and kits comprising spike-ins are encompassed within the scope of the disclosure herein, as are arrays that comprise probes complementary to the spike-ins. In the methods described herein, each of the spike-ins are present at a different concentration across a range of concentrations in order to determine the dynamic range of the microarray which is being used to evaluate a sample and to permit accurate identification of the linear dynamic range of the assay. In a further aspect, spike-ins are present at mass ratios spanning about 6 logs of magnitude when the mass ratio is determined by comparing the concentration of the spike-in to that of the total nucleic acid in the sample.

### BRIEF DESCRIPTION OF THE DRAWING

FIG. 1 shows a one color QC Report-Agilent Spike-Ins: Log (Signal) versus Log (Relative Concentration) Plot.

### DESCRIPTION

It is to be understood that the compositions and methods described herein are not limited to specific method steps, arrays, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like.

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA.

The term “nucleic acid” refers to a large molecule composed of nucleotide subunits and encompasses both RNA and DNA. A “nucleotide” refers to a subunit of a nucleic acid and has a phosphate group, a 5-carbon sugar and a nitrogen-containing base, as well as functional analogs (whether synthetic or naturally occurring) of such subunits which in the polymer form (as a polynucleotide) can hybridize with naturally occurring polynucleotides in a sequence-specific manner analogous to that of two naturally-occurring polynucleotides. Nucleotide subunits of deoxyribonucleic acids are deoxyribonucleotides, and nucleotide subunits of ribonucleic acids are ribonucleotides.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

An “oligonucleotide” generally refers to a nucleotide multimer of about 10 to 100 nucleotides in length, while a “polynucleotide” includes a nucleotide multimer having any number of nucleotides.

An “oligonucleotide probe” refers to a moiety made of an oligonucleotide or polynucleotide, comprising a discrete nucleic acid sequence complementary to a nucleic acid sequence present in a portion of a polynucleotide (i.e. a defined sequence) such as another oligonucleotide, or a target nucleic acid sequence, such that the probe will specifically hybridize to the target nucleic acid sequence under appropriate conditions. The term “defined sequence” refers to nucleic acids with known sequences complementary to sequences of oligonucleotides used as probes in the methods described herein. A “target” or “target sequence” is a nucleic acid whose identity or abundance is determined in a microarray experiment. In the methods described herein, the target sequences may be RNA sequences, for example.

The term “spike-in” refers to a defined sequence nucleic acid species (such as a RNA species, sequence or transcript) that is added to a sample during processing and used to assess the performance of a microarray. In some embodiments, the defined sequence nucleic acid comprises a sequence that is not likely to be found in the biological sample to be analyzed and is selected to have minimal self-hybridization and cross hybridization with other similar sequences in the set. Such spike in controls can be used to monitor microarray quality, in terms of dynamic range, reproducibility, etc. Different spike-in controls can be used to monitor different processes in a microarray analysis.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand “probe features” (i.e. an element or spot on an array that is made up of chemical moieties such as nucleic acid sequences, for example), or even more than one hundred thousand probe features, in an area of less than 20 cm² or even less than 10 cm². The term “probe feature” and “feature” are used interchangeably herein. For example, probe features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round probe features may have area ranges equivalent to that of circular probe features with the foregoing width (diameter) ranges. A given probe feature is made up of chemical moieties, e.g., nucleic acids, that bind to (e.g., hybridize to) the same target (e.g., target nucleic acid), such that a given feature corresponds to a particular target. At least some, or all, of the probe features have different sequences than some, or all of the other probe features on a given array. For example, when any repeats of each probe feature composition are excluded the remaining probe features may account for at least 5%, 10%, or 20% of the total number of features. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in some representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

“Hybridizing” and “binding”, with respect to polynucleotides, are used herein interchangeably.

A stringent hybridization and stringent hybridization wash conditions in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M followed by washes of 0.5×SSC and 0.1×SSC at room temperature, or by washed in 0.6×SSPE and 0.06×SSPE at elevated temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “predetermined” refers to an element whose identity or composition is known prior to its use. For example, a “predetermined temperature” is a temperature that is specified as a given temperature prior to use. An element may be known by name, sequence, molecular weight, its function, or any other attribute or identifier. As used herein, “automatic”, “automatically”, or other like term references a process or series of steps that occurs without further intervention by the user, typically as a result of a triggering event provided or performed by the user.

As used herein, the term “signal” refers to the detectable characteristic of a detectable molecule. Exemplary detectable characteristics include, but are not limited to: a change in the light adsorption characteristics of a reaction solution resulting from enzymatic action of an enzyme attached to a labeling probe acting on a substrate; the color or change in color of a dye; fluorescence; phosphorescence; radioactivity; or any other indicia that can be detected and/or quantified by a detection system being used.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination, which will perform those, or equivalent steps. Programming may be accomplished, for example, from a computer readable medium carrying necessary program code (such as a portable storage medium) or by communication from a remote location (such as through a communication channel).

A “memory” or “memory unit” refers to any device that can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box), which will provide additional protection to items contained in the package from exposure to the external environment. In the case of just a single array assembly a package may be that array assembly with some protective covering over the array assembly (which protective cover may or may not be an additional part of the array unit itself).

Methods for Measuring Dynamic Range of Arrays

The present description provides methods for measuring or monitoring the dynamic range of microarray applications. In embodiments, a plurality of defined sequence nucleic acids are hybridized to oligonucleotide probes present at predefined positions on one or more microarrays. Each defined sequence nucleic acid is different from other defined sequences in a given set of defined sequence nucleic acids, and each defined sequence is complementary to a corresponding oligonucleotide probe on the array, with each defined sequence being hybridized to the corresponding probe at a different concentration. Measuring the amount of hybridization of each defined sequence determines the dynamic range of the microarray.

In embodiments, the plurality of defined sequence nucleic acids comprise a mixture of in vitro synthesized sequences, such as a mixture of polyadenylated transcripts, or transcripts partially derived from adenovirus E1A transcripts. In aspects, the defined sequence nucleic acids comprise a mixture of about 10 sequences. In other aspects, the defined sequence compositions span a range of predefined concentrations that exceed the typical detection range of gene expression microarrays.

In embodiments, the plurality of defined sequence nucleic acids comprise nucleic acids premixed at concentrations spanning 6 logs in predefined logarithmic increments, such as, but not limited to, half log or one log, for example. In other embodiments, the plurality of defined sequence nucleic acids comprise positive control transcripts that are optimized to hybridize with corresponding oligonucleotides present on one or more microarrays. In aspects, optimization of these transcripts comprises minimizing self-hybridization and cross-hybridization of the positive control transcripts in the set or plurality.

The methods described herein are used in conjunction with kits for measuring and monitoring dynamic range for microarray applications. The kits include defined sequence nucleic acid compositions as described above, along with instructions for use of these sequences for determining dynamic array of a microarray. The kits also include reagents for amplifying and labeling defined sequence nucleic acid samples, reagents for hybridization, and reagents or buffers for use with the defined sequence, along with reagents for washing the microarray following hybridization.

Spike-In Compositions and Methods

The present disclosure provides compositions and methods for use in measuring the dynamic range in microarray analyses. In embodiments, this measurement involves the use of compositions for amplification and labeling of oligonucleotide probes used in microarray analyses. These compositions comprise a set of defined sequence nucleic acids that bind or hybridize, under the employed hybridization conditions, to control probes with complementary sequences at pre-defined positions within one or more arrays. In aspects, the compositions described herein are directed to the one-color method of analysis, and are referred to as one-color spike-in compositions. Since a gene microarray experiment is a multi-step procedure, small differences among samples, procedures, or user-induced variations may confound the microarray data. Confidence in the experimental data is increased when it is compared to control transcripts of known concentrations and ratios.

In one embodiment, a one color spike-in composition, according to the invention, provides a set of defined sequence nucleic acids for use as positive controls for monitoring a one color microarray workflow from sample amplification and labeling to microarray processing. In one aspect, a one color spike-in composition comprises a mixture of at least 10 in vitro synthesized, polyadenylated transcripts partially derived from the Adenovirus E1A transcriptome (Rosetta Inpharmatics, Seattle Wash.). The one color RNA spike-in composition comprises nucleic acids (e.g., transcripts) premixed at concentrations that span 6 logs in concentration when compared to the total nucleic acid in the sample and that differ from one another by either one log or half log increments. When the one color spike-in mixture is hybridized onto a one color microarray, the user can use the feature extraction software (such as that developed by Agilent, for example) to generate a quality control (QC) Report. This QC Report will identify the linear portion of the dynamic range of the microarray experiment, the high and low detection limits of the microarray as used in the experiment (as described in a copending application: Estimation Of Dynamic Range Of Microarray DNA Spike-In Data By Use Of Parametric Curve Fitting, incorporated by reference herein), as well as the reproducibility of the controls in the form of a % CV calculations for each of the spike-in treated samples (such as target nucleic acid samples mixed with the spike-in compositions).

In some embodiments, a spike-in composition comprises one or more defined sequence nucleic acids to form a set of nucleic acids. In some embodiments, the set includes at least 10 different sequences. Each of the defined sequence nucleic acids comprise a sequence designed as a sequence not likely to be complementary to any of the target sequences in the sample to be analyzed. Defined sequences may comprise a sequence also designed to minimize self hybridization and cross hybridization with other sequences in the set. In some embodiments, the defined sequences are designed for use in a one color microarray and are labeled with one detectable agent such as cyanine-3, cyanine-5, and phycoerythrin, for example.

In some embodiments, the spike-in compositions comprise a defined set of sequences each of which comprise a sequence are complementary to a different probe, and each of which comprise a nucleic acid encoding an adenovirus E1A sequence. The sequence of each defined sequence nucleic acid that is complementary to an oligonucleotide probe for are different from one another and in some embodiments are designed to minimize self hybridization and/or cross hybridization to other defined sequences in the set. In some embodiments, each of the defined sequences comprise the same E1A sequence. The E1A sequences are approximately 500 nt long, and are chosen to be part of the spike-in composition, because they are longer sequences similar to mammalian genes in composition. Using E1A transcripts in the spike-in control compositions makes the entire spike-in transcripts behave similarly to biological transcripts in the labeling reaction. In addition, because E1A is present in all the control compositions, all the spike-in transcripts behave similarly to each other.

The one color spike-in composition comprises a set of defined sequence nucleic acids, each present in a different concentration over a range of concentrations. In some embodiments, the range of concentrations exceeds that of the typical dynamic range of a microarray assay. The typical dynamic range of a microarray experiment ranges from 2.5 to 4 logs of signal; under some circumstances the dynamic range can go to 5 logs of signal. In some embodiments, the range of concentrations and/or the mass ratio of the set of defined sequence nucleic acids is at least 6 logs, wherein the mass ratio is the concentration each of the defined sequence nucleic acids as compared to the total concentration of nucleic acids in the sample. In some embodiments, the increment of change of concentration in the range of concentrations is at least 0.5 log and in some embodiments is at least 1 log.

In one aspect, concentrated one color spike-in stocks (i.e. components of a kit as described herein) are diluted with the dilution buffer provided in the kit. The controls are spiked-in directly to the sample comprising the target nucleic acid samples (for example, total RNA samples) prior to amplification and labeling to achieve the final relative amounts indicated in Table 1.

TABLE 1
Relative sample amounts in the concentrated stock

| | | | **Mass Ratio |
|---|---|---|---|
| | Log (Relative | Kit Stock | of E1A:total |
| RNA Spike-In Name* | Concentration) | Conc. (pg/ul) | RNA (1:x) |
| (+) E1A_r60_3 | 0.30 | 0.04 | 1:12,500,000,000 |
| (+) E1A_r60_a104 | 1.30 | 0.4 | 1:1,250,000,000 |
| (+) E1A_r60_a107 | 2.30 | 4 | 1:125,000,000 |
| (+) E1A_r60_a135 | 3.30 | 40 | 1:12,500,000 |
| (+) E1A_r60_a20 | 3.83 | 133 | 1:3,750,000 |
| (+) E1A_r60_a22 | 4.30 | 400 | 1:1,250,000 |
| (+) E1A_r60_a97 | 4.82 | 1333 | 1:375,000 |
| (+) E1A_r60_n11 | 5.30 | 4000 | 1:125,000 |
| (+) E1A_r60_n9 | 5.82 | 13333 | 1:37,500 |
| (+) E1A_r60_1 | 6.30 | 40000 | 1:12,500 |

*The Spike-In probe names listed in Agilent's Feature Extraction Software v8.5 may or may not contain the prefix (+) E1A.
**The E1A mass to total RNA mass is based on the dilutions recommended in this protocol. The dilutions can be varied by the user to adjust for the mRNA content of the Total RNA being sampled.

The one color spike-in controls are then labeled together with the nucleic acid samples of interest being analyzed (optionally during or after being amplified). After hybridization to complementary probes on the microarray, the signal intensities of the detectable label (e.g., green signal) for each spike-in transcript can be used to monitor the sample amplification and labeling and microarray processing procedures used in the experiment, which are independent from the quality of the starting RNA sample. These controls also allow the measurement of the dynamic range of the assay, accounting for all steps in the procedure, including, but not limited to, amplification, labeling, hybridization, and data processing.

Kits

In certain aspects, kits according to the description herein include one or more of: a composition comprising a set of defined sequence nucleic acids that hybridize to complementary sequences on one or more microarrays, all or some of the components of amplification reagents, cRNA cleanup components, hybridization reagents, or wash reagents. For example, the kits can include components from an Agilent Low RNA Input Linear Amplification Kit (PN 5184-3523); Gene Expression Hybridization Kit (PN 5188-5242) and Gene Expression Wash Pack (PN 5188-5327), all available from Agilent Technologies, Inc. (Palo Alto, Calif.). In embodiments, the kits also include instructions for the use of various kit components or information about the validation of the compositions comprising a set of defined sequence nucleic acids for the array according to the methods described herein.

Arrays

The present description also provides nucleic acid microarrays, as described herein. The subject arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array.

The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another. An exemplary array is described in U.S. Patent Publication No. 20050095596, which is incorporated herein by reference.

The arrays as described herein comprise a set of oligonucleotide probes each of the set of probes present at a predefined position on one or more microarrays. In some embodiments, the set includes at least 10 different sequences. The sequences are designed as sequences not likely to be complementary to any of the target sequences in the sample to be analyzed. In some embodiments, each of the probes are complementary to a different defined sequence nucleic acid in the set. In some embodiments, each of the defined nucleic acids comprise a sequence that is complementary to a probe and is different from the other defined nucleic acids in the set and the E1A transcriptome.

Each of the set of oligonucleotide probes are located in predetermined positions in one or more microarrays. When more than one microarray is employed, each microarray may include all of the oligonucleotide probes in the set or may include one, two, three, etc, and up to all of the oligonucleotide probes in the set. The oligonucleotide probes can be located in different patterns as described above.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Methods other than drop deposition can be used for fabrication, as previously described.

The arrays as described herein can be used in a variety of different microarray applications, including gene expression experiments and genomic analysis. In using an array, the array will typically be exposed to a sample (for example, a fluorescently labeled analyte, such as from a sample containing messenger RNA) and the array then read. Reading of the array may be accomplished by illuminating the array and reading the location and intensity of resulting fluorescence at each feature of the array to detect any binding complexes on the surface of the array. For example, a scanner may be used for this purpose that is similar to the AGILENT MICROARRAY SCANNER available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

Data Analysis

Once the signals are read, the data can be analyzed using a variety of methods. In some embodiments, the user can use the feature extraction software (such as that developed by Agilent, for example) to generate a quality control (QC) report. This QC report will identify the linear portion of the dynamic range of the microarray experiment, the high and low detection limits of the microarray as used in the experiment (as described in a copending application: Estimation Of Dynamic Range Of Microarray DNA Spike-In Data By Use Of Parametric Curve Fitting, incorporated by reference herein), as well as the reproducibility of the controls in the form of a % CV. Calculations for each of the spike-in treated samples (such as target nucleic acid samples mixed with the spike-in compositions). The term “CV” refers to coefficient of variation, which is defined as the ratio of the standard deviation to an average value. % CV functions as a metric that can be used to assess the reproducibility or quality of a microarray. The Quality control report based on the detection of the compositions as described herein provides the assessment of the performance of the array in the particular experiment for criteria including, but not limited to signal to noise, dynamic range, linearity of response, and background. Other methods of data analysis are known to those of skill in the art and can be readily employed using standard methods.

### EXAMPLES

Preparation of RNA Spike-In Mix for Target Labeling

Generally, stock solutions of the spike-in compositions are vortexed vigorously, and heated at 37° C. for 5 minutes, and vortexed once more. Tubes are briefly centrifuged to drive contents to the bottom of the tube prior to opening. Table 2 provides the amount of spike-in composition required for a range of total RNA or 0.2 μg of polyA mRNA input amounts. These are diluted such that 1 μl is added for every 100 ng of total RNA that is labeled.

TABLE 2
Sample dilution for Spike-In Composition
in Kit for cyanine 3 labeling

| | | | | | Spike-In Mix |
|---|---|---|---|---|---|
| Starting Amount of RNA | | | | | volume to |
| Total | Maximum | | | | be used in |
| RNA | Volume | Serial Dilutions | | | each labeling |
| (ng) | of RNA (ul) | First | Second | Third | reaction (ul) |
| 200 | 8.3 | 1:20 | 1:25 | 1:10 | 2 |
| 300 | 7.3 | 1:20 | 1:25 | 1:10 | 3 |
| 400 | 6.3 | 1:20 | 1:25 | 1:10 | 4 |
| 500 | 5.3 | 1:20 | 1:25 | 1:10 | 5 |
| 600 | 4.3 | 1:20 | 1:25 | 1:10 | 6 |
| 700 | 6.8 | 1:20 | 1:25 | 1:5 | 3.5 |
| 800 | 6.3 | 1:20 | 1:25 | 1:5 | 4 |
| 900 | 5.8 | 1:20 | 1:25 | 1:5 | 4.5 |
| 1000 | 5.3 | 1:20 | 1:25 | 1:5 | 5 |

Standard conditions for maintaining an RNAse-free environment should be used.

For example, to prepare the appropriate spike-in dilutions for 200 ng of total RNA starting sample: spike-in compositions (in stock or concentrate form) are vigorously vortex thawed and then heated at 37° C. for 5 minutes, and then vortexed, centrifuged and spun down. 2 μl of this stock solution is mixed with a specific volume of dilution buffer provided in the kits as described herein (a 1:20 dilution). The mixture is thoroughly vortex and spun down, making the First Dilution. A Second Dilution is prepared by mixing 2 μl of the spike-in stock solution with a specific volume of dilution buffer (a 1:25 dilution). A Third Dilution (1:10) is similarly prepared. The Third Dilution is added to 200 ng of sample (containing the target nucleic acid, such as total RNA, for example). The mixture is then labeled with a fluorophore, such as cyanine-3 (Cy3), for example. Sample mixtures may be amplified before or after labeling, using low input fluorescent linear amplification

Sample Data

Sample data shown in Table 3 were obtained following amplification, labeling, and hybridization of the spike-in controls to an appropriate microarray, followed by data extraction with Agilent Feature Extraction v8.5 software. The data are reported as part of the Agilent QC Report generated by said software.

TABLE 3
One Color QC Report - Agilent Spike-In Signal Statistics
Agilent SpikeIns Signal Statistics

| | Log(Relative | Log(Median | | |
|---|---|---|---|---|
| Probe Name | Conc.) | Proc. Sig.) | % CV | StdDev |
| (+)E1A_r60_3 | 0.30 | 0.15 | 105.74 | 0.55 |
| (+)E1A_r60_a104 | 1.30 | 0.30 | 89.52 | 0.61 |
| (+)E1A_r60_a107 | 2.30 | 1.08 | 36.28 | 0.19 |
| (+)E1A_r60_a135 | 3.30 | 2.24 | 7.76 | 0.03 |
| (+)E1A_r60_a20 | 3.83 | 2.59 | 7.40 | 0.03 |
| (+)E1A_r60_a22 | 4.30 | 3.24 | 5.99 | 0.03 |
| (+)E1A_r60_a97 | 4.82 | 3.80 | 5.94 | 0.03 |
| (+)E1A_r60_n11 | 5.30 | 4.32 | 6.91 | 0.03 |
| (+)E1A_r60_n9 | 5.82 | 4.75 | 4.86 | 0.02 |
| (+)E1A_r60_1 | 6.30 | 4.81 | 5.82 | 0.02 |

For each sequence of spike-in, Table 3 shows the Probe Name, the median Processed Signal (median of LogProcessedSignal), % CV, and the standard deviation (of LogProcessedSignals).

RNA Spike-In Mix with Total RNA

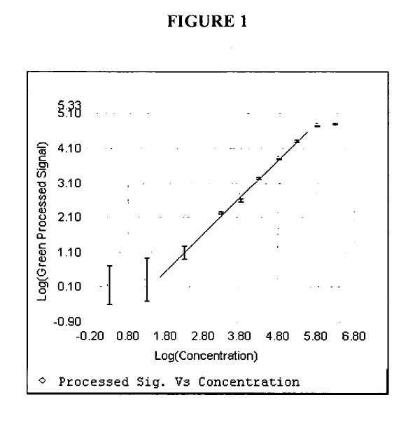

Data in FIG. 1 were generated by amplifying and labeling Agilent One Color RNA Spike-In transcripts with total RNA from the MG63 osteosarcoma cell line (Ambion) in the presence of cyanine 3-CTP starting with 200 ng total RNA. Cyanine 3-labeled targets were hybridized to Agilent Human 1A (v2, part number G4110B) oligo microarrays and cyanine 3 signals were calculated. Data representing the green signal for each spike-in transcript is plotted against the log of the relative concentration. The data in FIG. 1 shows the relative concentrations of the different transcripts (relative to each other), the log Signals from each (median), and reproducibility of those signals. This data is also presented in a table in the One Color QC Report, which also details the % CV and standard deviation for each of the Spike-In transcripts, as shown in Table 3 above. The plot in FIG. 1 is used to calculate the detection limit and dynamic range, which are shown in Table 4.

The dynamic range of the experiment measured by the spike-in controls can be determined because transcripts present at high concentration are present beyond the typical range of biological transcripts when the transcript mix is used as recommended and transcripts present at low concentration are present at levels lower than that typically detectable by gene expression assays when used as recommended. The concentrations are as provided in Table 1.

As seen in FIG. 1, at high signal levels the error bars are small since the scanner reaches saturation at this point. At low signal levels the error bars are visible because the signal is dropping into the background noise. The signal level at the top of the error bars of the features with the lowest signals provides a rough estimate of the lower limit of detection, as calculated from the % CV plot in FIG. 1, and shown in Table 4.

The Agilent Feature Extraction v8.5 software also generates Spike-In concentration response statistics from the data generated by the spike-in controls as presented in Table 4, which provides the detection limit and dynamic range.

TABLE 4
One Color QC Report -
Agilent Spike-In Concentration-Response Statistics
Agilent Spike-In Concentration-Response Statistics
Linear Range Statistics:

| | |
|---|---|
| Low Signal | 0.40 |
| High Signal | 4.60 |
| Low Relative Concentration | 1.67 |
| High Relative Concentration | 5.58 |
| Slope | 1.07 |
| R\hat{ }2 Value | 1.00 |

Detection Limit Statistics

| | |
|---|---|
| Saturation Point | 4.82 |
| Low Threshold | 0.07 |
| Low Threshold Error | 0.85 |

Table 4 presents the values calculated from the log signal vs. log concentration plot shown in FIG. 1. All of the statistics in the table above are calculated using a parameterized sigmoidal curve fit to the data shown in the plot, as indicated in a copending application, entitled “Estimation Of Dynamic Range Of Microarray DNA Spike-In Data By Use Of Parametric Curve Fitting”, and incorporated herein by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The claims are:

1. A method for measuring or monitoring a dynamic range of a microarray application, comprising
hybridizing a plurality of defined sequence nucleic acids to a plurality of oligonucleotide probes present at predefined positions on one or more microarrays, and
measuring the amount of hybridization of each of the defined sequence nucleic acids to determine the dynamic range of the microarray,
wherein each of the defined sequence nucleic acids comprise a sequence different from one another, with each defined sequence being complementary to a corresponding oligonucleotide probe present on the array, and further wherein each defined sequence is hybridized to its corresponding probe at a different concentration.

2. The method of claim 1, wherein the plurality of defined sequence nucleic acids comprises a mixture of in vitro synthesized sequences.

3. The method of claim 1, wherein the plurality of defined sequence nucleic acids comprises a mixture of polyadenylated transcripts.

4. The method of claim 3, wherein the polyadenylated transcripts are partially derived from Adenovirus E1A transcript.

5. The method of claim 1, wherein the plurality of defined sequence nucleic acids comprises a mixture of about 10 sequences.

6. The method of claim 1, wherein the plurality of defined sequence nucleic acids comprise nucleic acids that span a predefined range of concentrations.

7. The method of claim 6, wherein the predefined range of concentrations exceeds the typical detection range of gene expression assays.

8. The method of claim 6, wherein the plurality of defined sequence nucleic acids comprise nucleic acid sequences premixed at concentrations spanning 6 logs in increments of one log.

9. The method of claim 8, wherein the plurality of defined sequence nucleic acids comprise nucleic acid sequences premixed at concentrations spanning 6 logs in increments of half log.

10. The method of claim 6, wherein the plurality of defined sequence nucleic acids comprise nucleic acid sequences premixed at concentrations spanning 6 logs in predefined logarithmic increments.

11. The method of claim 1, wherein the plurality of defined sequence nucleic acids comprises a set of positive control transcripts optimized to hybridize with a plurality of corresponding oligonucleotide probes present on one or more microarrays.

12. The method of claim 11, wherein the optimization of the positive control transcripts comprises minimizing self-hybridization and cross-hybridization of the positive control transcripts.

13. A kit for measuring and monitoring dynamic range in a microarray application, comprising:
a composition comprising a plurality of defined sequence nucleic acids, wherein each of the defined sequence nucleic acids comprise sequences different from one another, with each defined sequence being complementary to a corresponding oligonucleotide probe present on the array, and further wherein each defined sequence is hybridized to its corresponding probe at a different concentration; and
a set of instructions for a microarray experiment using the composition.

14. The kit of claim 13, further comprising:
reagents for amplification and labeling of defined sequence nucleic acids and RNA samples;
reagents for hybridization of said samples to oligonucleotide probes on a microarray;
reagents or buffers for use with defined sequence nucleic acids; and
reagents for washing of said microarray.
